(19) United States
(12) Patent Application Publication
(10) Pub. No.: US 2026/0183024 A1
(43) Pub. Date: Jul. 2, 2026

(54) Advanced T-bar for intraoperative assessment of coronal alignment in spine surgery

(71) Applicant: NexSpine Orthopedics Trust, St. Petersburg, FL (US)

(72) Inventor: Jose Manuel Sarmiento, St. Petersburg, FL (US)

(21) Appl. No.: 19/004,517

(22) Filed: Dec. 30, 2024

(51) Int. Cl. A61B 17/70

(52) U.S. Cl. CPC A61B 17/7083

(57) ABSTRACT

According to an aspect of the present invention, there is provided stainless steel, round rod in a “T” configuration, used for the effective intraoperative assessment of coronal alignment using fluoroscopy during spine surgery, comprising: a vertical stainless steel rod; a horizontal stainless steel rod; and a disposable level gauge, wherein: the two stainless steel round rods are cut to appropriate dimensions and welded together in a “T” configuration; and a pocket is cut into the horizontal rod for the placement of a round level gauge, such that the level gauge helps achieve optimal orthogonal placement of the T-bar over the spine.

### CROSS-REFERENCE TO RELATED APPLICATIONS

This application claims the benefit of priority of U.S. Provisional Patent Application No. #63/692,772, filed Sep. 10, 2024.

### BACKGROUND

The invention relates generally to an advanced T-bar for intraoperative assessment of coronal alignment in spine surgery.

Obtaining optimal coronal alignment of the spine after spinal surgery is critical to achieving excellent clinical outcomes and preventing postoperative patient disability. Coronal malalignment is defined as lateral deviation of the C7 plumb line with substantial displacement from the midline of the pelvis by more than 30 mm. However, there is no standard method for assessing intraoperative coronal spinal alignment, and this task is particularly challenging for the following reasons: First, patients with advanced spinal deformities may have large and rigid curves that are often associated with concomitant pelvic obliquity. This makes prone positioning difficult before surgery and complicates the assessment of long spinal fusion constructs to the pelvis, as the pelvis is not inherently balanced. Second, long spinal fusion constructs make it difficult to evaluate appropriate spinal balance during surgery due to the limitations of the fluoroscopic image diameter and the inability to view the entire spine as a whole. Obtaining long radiographs intraoperatively to visualize the entire spinal fusion construct is challenging because most hospital radiology departments lack long radiograph capabilities, and a radiolucent operating table with long-length cassette capability is required.

Currently, there are several radiographic solutions for assessing coronal alignment of the spine. Unfortunately, many radiographic techniques for assessing coronal alignment can only be used postoperatively. Examples of classic radiographic parameters for measuring coronal malalignment include the C7 coronal vertical axis (C7-CVA), orbital coronal vertical axis (ORB-CVA), and odontoid coronal vertical axis (OD-CVA). Newer parameters have been proposed that use the L5 pedicles as the reference and determine the position of a plumb line drawn from the odontoid tip (OD-L5), the midpoint between the medial orbital rims (ORB-L5), and the C7 centroid (C7-L5) relative to the L5 pedicles. However, a T-bar with a level gauge permits reliable assessment of real-time, intraoperative coronal alignment of the spine, allowing for intraoperative technique modifications before the completion of surgery. Furthermore, our T-bar surgical instrument is a practical, inexpensive solution to assess intraoperative coronal spinal alignment. It is easy to use, time-efficient, and does not require cost-prohibitive, expensive new technology. Finally, our T-bar with a level gauge is made from stainless steel, which provides durability and ease of handling during surgery, and it utilizes a disposable level gauge that allows accurate positioning of the tool over the patient's pelvis and sacrum. Importantly, the level gauge feature also helps evaluate pelvic obliquity, which can confound the evaluation of coronal spinal alignment because the sacrum is not balanced over the spine in these cases.

### SUMMARY OF INVENTION

Therefore, it would be desirable to have a device that reliably assesses intraoperative coronal spinal alignment during spine surgery, one that is intuitive, easy to use, and highly visible on x-ray without the need for advanced 3D imaging. Furthermore, it would also be desirable to have a device that is sufficiently large, lightweight, and durable. Additionally, it would be desirable to have a device that is portable and does not encumber the patient, the surgeon, or the operating room staff. Therefore, a need currently exists in the industry for a device that effectively assesses intraoperative coronal alignment and pelvic obliquity during spine surgery.

Similarly, it would be desirable to have an associated method that can physically establish a cranial plumb line reference mark during surgery and apply it for any patient undergoing spine surgery in the prone position from a posterior approach. Thus, a need currently exists in the industry for a process that effectively assesses intraoperative coronal alignment in spine surgery.

Disclosed is an advanced T-bar for the intraoperative assessment of coronal alignment in spine surgery, composed of the following components: a disposable level gauge and two stainless steel round rods. These components are related as follows: the stainless steel rods are assembled to form a T-bar, and a level gauge is inserted at its base. The associated method consists of the following steps: a pocket is cut into a horizontal stainless steel round rod to insert a disposable level gauge. A separate vertical stainless steel round rod is welded to the horizontal rod to form a “T”-shaped configuration.

The device may also include one or more of the following: 1) a level gauge is not necessarily needed at the base of the T-bar; however, having a level gauge in place will simplify the process of appropriately leveling the T-bar over the pelvis and orthogonal to the spine; 2) a level gauge may also be placed at the base or the end of the vertical stainless steel round rod to further assist in achieving proper positioning for accurate intraoperative spinal coronal alignment; 3) the round rods that comprise the T-bar may be made of several other materials besides stainless steel, including titanium, aluminum, carbon fiber, composite materials, brass, copper, fiberglass, steel (non-stainless), plastics (e.g., PVC, nylon, polyethylene), iron, and brass; 4) a laser line level may be used in addition to or instead of a level gauge on the T-bar. A laser line level tool can be combined with a spirit level and/or plumb bob with a laser to display an accurately horizontal or vertical illuminated line on a surface, in this case, a patient's pelvis and back. Similarly, the associated method may also include one or more of the following steps:
1. The appropriate stainless steel grades that may be used include SAE 316 (Austenitic Stainless Steel), SAE 304, 304L, and 305.
2. Cutting stainless steel round rods may be performed manually with a hacksaw, power shears, hand grinder or circular saw.
3. If a lathe is used for cutting stainless steel round rods, the following set up is needed: 1) use carbide inserts suitable for stainless steel; 2) position the round bar securely between the lathe's headstock and tailstock; 3) apply the tool to the bar while turning, ensuring a clean cut.
4. Cutting the level gauge pocket on the horizontal stainless steel round rod using Computer Numerical Code (CNC) machining requires using a small ball nose R0.5 mm. Alternatively, a Milling Cutter R3 mm can make the pocket for the level gauge on the horizontal stainless steel rod.
5. Welding methods for conjoining the two stainless steel rods include: Tungsten Inert Gas welding, Metal Inert Gas welding, shielded metal arc welding, and flux-cored arc welding. ER308L or ER316L consumables are compatible with austenitic stainless steels (e.g., 304, 316).
6. Post-weld treatments include pickling (removing oxide layers) or passivation (enhancing corrosion resistance).
7. Assembly of the stainless steel rods with level gauge may be done with adhesive or Double Tap M3.

We describe an advanced T-bar for the intraoperative assessment of coronal spinal alignment in spine surgery, composed of the following components: a disposable level gauge and two stainless steel round rods that are welded together in a “T” configuration. Proper use of this device is based on the following core steps: the T-bar is placed on a patient's back during surgery in an upside-down “T” configuration. The horizontal rod of the T-bar should ideally lie over the patient's pelvis, roughly at the level of the posterior superior iliac spine and cranial to the femoral heads. The vertical rod of the T-bar should bisect the sacrum. Intraoperative fluoroscopy is then performed, and coronal x-rays are used to compare the alignment of the T-bar against the patient's thoracolumbar spine. In this way, the T-bar acts as a surgical tool to represent a cranial plumb line that helps surgeons achieve optimal coronal alignment, especially during spinal deformity surgeries. If there is misalignment between the spine and the vertical rod of the T-bar, reduction maneuvers can be performed to rectify the spinal coronal alignment. Ultimately, at the conclusion of these steps, optimal intraoperative coronal spinal alignment will be achieved after the vertical T-bar rod crosses the vertebral body of either C7 or T1.

The device is a rod that could be round or square-shaped, formed in a “T” configuration from a radio-opaque material used for the effective intraoperative assessment of coronal alignment using fluoroscopy during spine surgery. We chose to use round stainless steel rods for the prototype because of the durability, high visibility on fluoroscopy, and ease of handling that this material affords, but other materials that can be used include titanium, aluminum, carbon fiber, composite materials, brass, copper, fiberglass, steel (non-stainless), plastics with radio-opaque markers (e.g., PVC, nylon, polyethylene), iron, and brass. This device ensures proper coronal alignment of the spine during spinal fusion surgeries, particularly after spinal instrumentation and implants have been placed. The core components of the invention are stainless steel rods and a disposable level gauge (other synonyms include bubble gauge, level vial, spirit-level, and bullseye level), which are generally configured as follows: two stainless steel round rods are cut to their appropriate dimensions and welded together in a “T” configuration. A pocket is cut into the horizontal rod for the placement of a round level gauge. The level gauge helps achieve optimal orthogonal placement of the T-bar over the spine, and it helps evaluate for pelvic obliquity, which can confound the evaluation of coronal spinal alignment because the sacrum is not balanced over the spine in these cases.

This disclosure will now provide a more detailed and specific description that will refer to the accompanying drawings. The drawings and specific descriptions of the drawings, as well as any specific or alternative embodiments discussed, are intended to be read in conjunction with the entirety of this disclosure. The Advanced T-bar for intraoperative assessment of coronal alignment in spine surgery may, however, be embodied in many different forms and should not be construed as being limited to the embodiments set forth herein; rather, these embodiments are provided by way of illustration only and so that this disclosure will be thorough, complete and fully convey understanding to those skilled in the art.

### BRIEF DESCRIPTION OF THE DRAWINGS

FIG. 1. Illustrates an advanced T-bar.

FIG. 2 depicts sample disposable level gauges and specifications for the cut-out pocket.

FIG. 3 is a diagram of the welding process for joining both stainless steel rods together.

FIG. 4 is a depiction of the vertical stainless steel rod.

FIG. 5 is a depiction of the horizontal stainless steel rod including a pocket for a level gauge.

### DETAILED DESCRIPTION

The present invention is directed to advanced T-bar device for intraoperative assessment of coronal alignment in spine surgery. The device is a stainless steel, round rod in a “T” configuration, used for the effective intraoperative assessment of coronal alignment using fluoroscopy during spine surgery. This device ensures proper coronal alignment of the spine during spinal fusion surgeries, particularly after spinal instrumentation and implants have been placed. The core components of the invention are stainless steel rods and a disposable level gauge (other synonyms include bubble gauge, level vial, spirit-level, and bullseye level), which are generally configured as follows: two stainless steel round rods are cut to their appropriate dimensions and welded together in a “T” configuration. A pocket is cut into the horizontal rod for the placement of a round level gauge. With respect to the device, it should be further noted that the level gauge helps achieve optimal orthogonal placement of the T-bar over the spine. The stainless steel rods allow for ease of use and provide excellent visibility on fluoroscopy.

Regarding the associated method, the following core steps are followed: the T-bar is placed on a patient's back during surgery in an upside-down “T” configuration. The horizontal rod of the T-bar should ideally lie over the patient's pelvis, roughly at the level of the posterior superior iliac spine and cranial to the femoral heads. The vertical rod of the T-bar should bisect the sacrum. Intraoperative fluoroscopy is then performed, and coronal x-rays are used to compare the alignment of the T-bar against the patient's thoracolumbar spine. In this way, the T-bar acts as a surgical tool to represent a cranial plumb line that helps surgeons achieve optimal coronal alignment, especially during spinal deformity surgeries. If there is misalignment between the spine and the vertical rod of the T-bar, reduction maneuvers can be performed to rectify the spinal coronal alignment. Ultimately, at the conclusion of these steps, optimal intraoperative coronal spinal alignment will be achieved after the vertical T-bar rod crosses the vertebral body of either C7 or T1.

In its most complete form, the device is made up of the following components: a disposable level gauge and two stainless steel round rods. These components are related as follows: the stainless steel rods are assembled to form a T-bar, and a level gauge is inserted at its base. It should further be noted that the device may also include one or more of the following:
1) a level gauge is not necessarily needed at the base of the T-bar; however, having a level gauge in place will simplify the process of appropriately leveling the T-bar over the pelvis and orthogonal to the spine; 2) a level gauge may also be placed at the base or the end of the vertical stainless steel round rod to further assist in achieving proper positioning for accurate intraoperative spinal coronal alignment; 3) the round rods that comprise the T-bar may be made of several other materials besides stainless steel, including titanium, aluminum, carbon fiber, composite materials, brass, copper, fiberglass, steel (non-stainless), plastics with radio-opaque markers (e.g., PVC, nylon, polyethylene), iron, and brass; 4) a laser line level may be used in addition to or instead of a level gauge on the T-bar. A laser line level tool can be combined with a spirit level and/or plumb bob with a laser to display an accurately horizontal or vertical illuminated line on a surface, in this case, a patient's pelvis and back.

The most complete form of performing the method associated with the disclosed device consists of the following steps: 1) a pocket is cut into a horizontal stainless steel round rod to insert a disposable level gauge; 2) a separate vertical stainless steel round rod is welded to the horizontal rod to form a “T” shaped configuration. It should further be noted that: 1) The appropriate stainless steel grades that may be used include SAE 316(Austenitic Stainless Steel), SAE 304, 304L, and 305; 2) Cutting stainless steel round rods may be performed manually with a hacksaw, power shears, hand grinder or circular saw; 3) Cutting the level gauge pocket on the horizontal stainless steel round rod using Computer Numerical Code (CNC) machining requires using a small ball nose R0.5 mm. Alternatively, a Milling Cutter R3 mm can make the pocket for the level gauge on the horizontal stainless steel rod; 3) Welding methods for conjoining the two stainless steel rods include: Tungsten Inert Gas welding, Metal Inert Gas welding, shielded metal arc welding, and flux-cored arc welding. ER308L or ER316L consumables are compatible with austenitic stainless steels (e.g., 304, 316); and assembly of the stainless steel rods with level gauge may be done with adhesive or Double Tap M3.

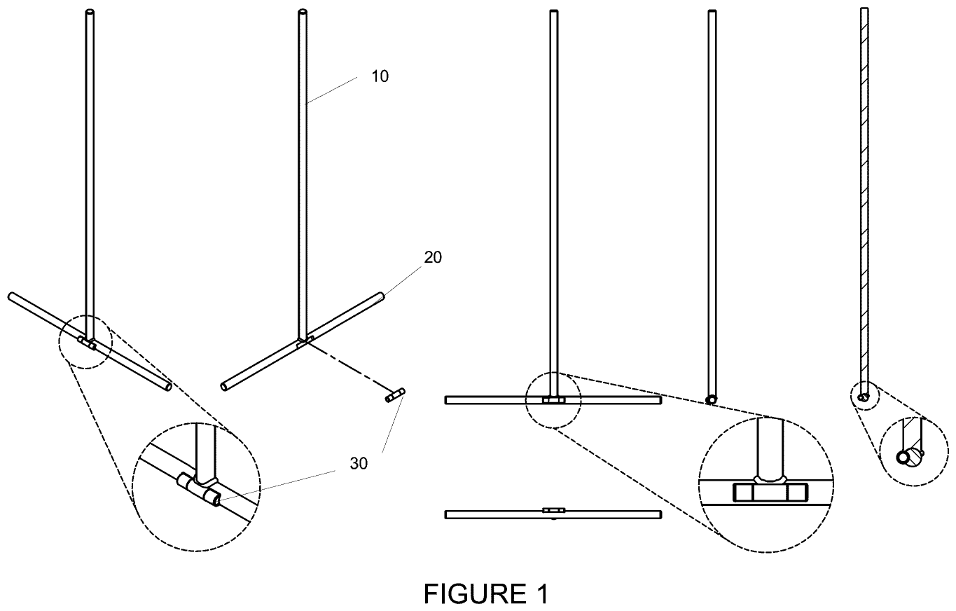

The following reference numerals are illustrated in FIGS. 1-5:
10—Vertical Rod
12—Notch for joining the rods in T configuration
20—Horizontal Rod
22—Cut Groove for installing level gauge
30—Level Gauge.

FIG. 1 shows an angled front views of fully assembled advanced T-bar with closeup of the level gauge. There are also design specifications of the advanced T-bar from front, side, and inferior angles.

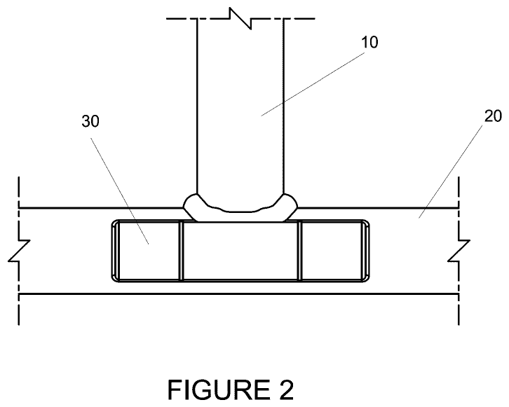

FIG. 2 depicts sample disposable level gauges and front-view specifications for the cut-out pocket on the horizontal stainless steel rod.

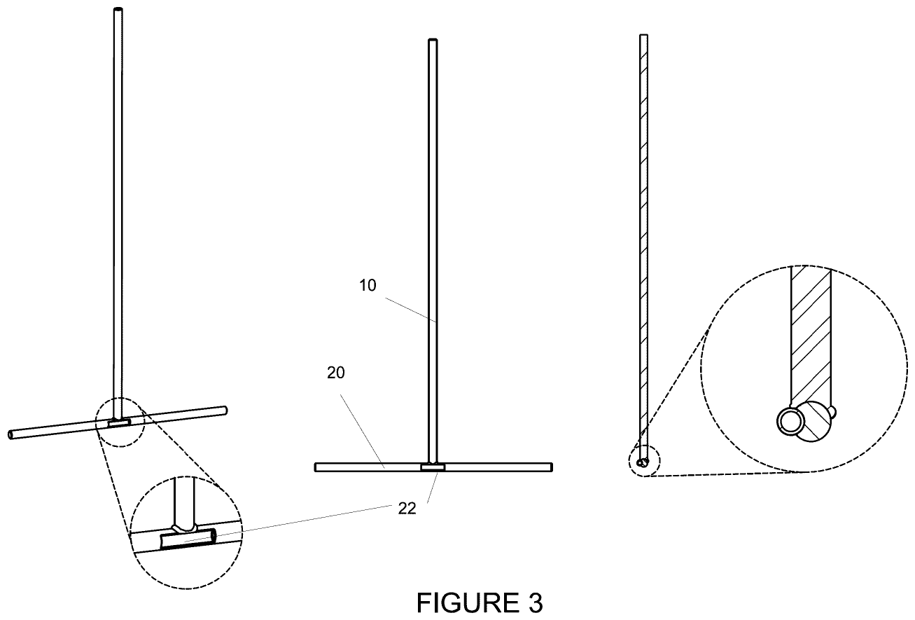

FIG. 3 is a diagram of the welding process for joining both stainless steel rods together and includes a rendering of the T-bar from angled front views without the level gauge in place. The drawings depict the welding sites necessary to join the stainless steel rods to form the Advanced T-bar.

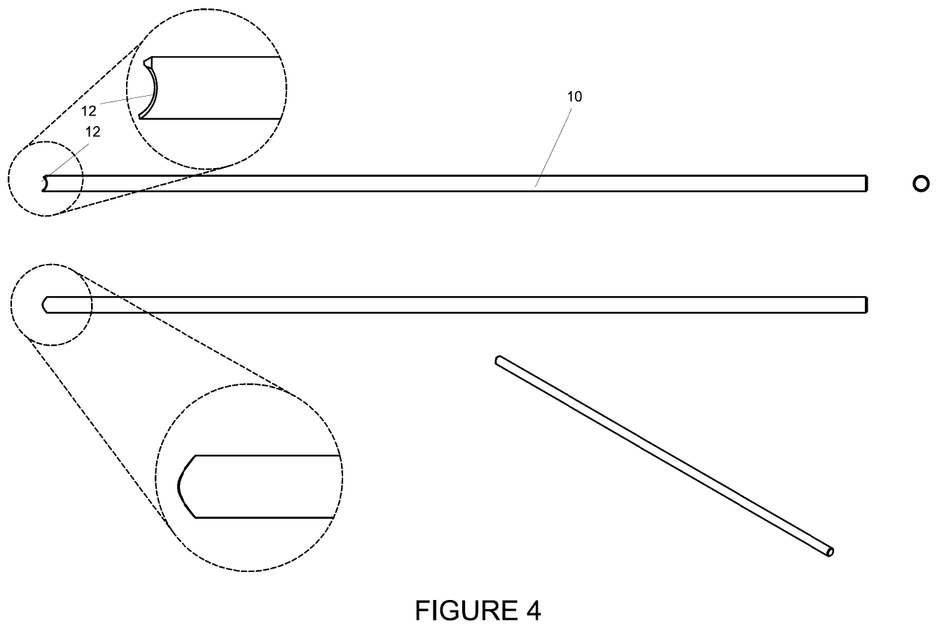

FIG. 4 is a rendering and depiction of the vertical stainless steel rod, and it illustrates the cut needed to join it to the second horizontal rod.

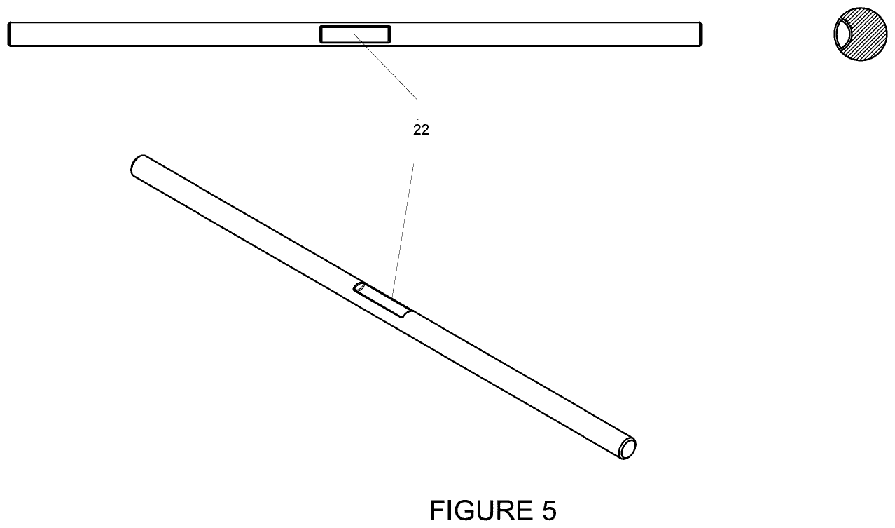

FIG. 5 is a rendering and depiction of the horizontal stainless steel rod including a highlight specification of the cut needed to form the pocket for the disposable level gauge.

The illustrations of embodiments described herein are intended to provide a general understanding of the structure of various embodiments, and they are not intended to serve as a complete description of all the elements and features of apparatus and systems that might make use of the structures described herein. Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Similar numerals designate similar elements among the several figures. Certain proportions thereof may be exaggerated, while others may be minimized. Accordingly, the specification and drawings are to be regarded in an illustrative rather than a restrictive sense. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed. It is indeed intended that the scope of this disclosure should be determined by a proper legal interpretation and construction of the disclosure, including equivalents, as understood by those of skill in the art relying upon the complete disclosure present at the time of filing.

What is claimed is:

1. A stainless steel, round rod in a “T” configuration, used for the effective intraoperative assessment of coronal alignment using fluoroscopy during spine surgery, comprising:
a vertical stainless steel rod;
a horizontal stainless steel rod; and
a disposable level gauge, wherein:
the two stainless steel round rods are cut to appropriate dimensions and welded together in a “T” configuration; and
a pocket is cut into the horizontal rod for the placement of a round level gauge, such that the level gauge helps achieve optimal orthogonal placement of the T-bar over the spine.

2. A method, comprising:
placing the T-bar of claim 1 on a patient's back during surgery in an upside-down “T” configuration, wherein the horizontal rod of the T-bar lies over the patient's pelvis, roughly at the level of the posterior superior iliac spine and cranial to the femoral heads and the vertical rod of the T-bar bisects the sacrum;
performing intraoperative fluoroscopy is then performed and usingcoronal x-rays are used to compare the alignment of the T-bar against the patient's thoracolumbar spine;
using the T-bar as a surgical tool to represent a cranial plumb line that helps surgeons achieve optimal coronal alignment.

3. The method of claim 2, further comprising:
Determining if there is misalignment between the spine and the vertical rod of the T-bar; and
Performing one or more reduction maneuvers to rectify the spinal coronal alignment.

4. The method of claim 3, further comprising:
Achieving optimal intraoperative coronal spinal alignment after the vertical T-bar rod crosses the vertebral body of either C7 or T1.
